(19) United States
(12) Patent Application Publication
(10) Pub. No.: US 2025/0345255 A1
(43) Pub. Date: Nov. 13, 2025

(54) COMPOSITIONS FOR IMPROVING NAIL HEALTH

(71) Applicant: L'ORÉAL, Paris (FR)

(72) Inventors: Sam MORRISON, Piscataway, NJ (US); Ramakrishnan Hariharan, Springfield, NJ (US); I-Fan Hsieh, Scotch Plains, NJ (US)

(73) Assignee: L'ORÉAL, Paris (FR)

(21) Appl. No.: 19/206,544

(22) Filed: May 13, 2025

Related U.S. Application Data
(60) Provisional application No. 63/646,219, filed on May 13, 2024.

(30) Foreign Application Priority Data
Jun. 20, 2024 (FR) 2406626

(51) Int. Cl. A61K 8/34; A61K 8/67; A61K 8/92; A61Q 3/00

(52) U.S. Cl. CPC A61K 8/347; A61K 8/675; A61K 8/922; A61Q 3/00; A61K 2800/30; A61K 2800/31

(57) ABSTRACT

Techniques for improving nail health of a user may be provided. The method may include providing a composition including an oil phase. The oil phase may include a vasodilator. The vasodilator may be present in a concentration (C_{V}) of, e.g., 0<C_{V}≤5% by weight of the composition. The vasodilator may be a single vasodilator (such as vanillyl butyl ether). The vasodilator may include a plurality of vasodilators. The oil phase may include a non-volatile oil. The non-volatile oil may include one or more vegetable oils, which may be seed oils. The non-volatile oil may be present in a concentration (C_{NVO}) of C_{NVO}≥90% by weight of the composition. The oil phase may include a preservative and an antioxidant. The composition may be in the form of a liquid oil, a thickened oil, or a paste.

### CROSS-REFERENCE TO RELATED APPLICATIONS

The present application claims priority to U.S. Provisional Patent App. No. 63/646,219 filed 13 May 2024, and French Patent App. No. FR 2406626 filed Jun. 20, 2024, the contents of which are each incorporated by reference herein in its entirety.

### TECHNICAL FIELD

The present disclosure is drawn to cosmetic compositions for improving nail health.

### BACKGROUND

Having healthy nails and skin around the nails is desirable. Healthy nails have a healthy color, they have a relatively smooth surface that has a gentle curve, they do not break easily, and they have a healthy cuticle and are surrounded by healthy skin.

Use of conventional nail cosmetics and nail cosmetic removers may be a factor in reduced nail health; for example, if the nail cosmetic remover utilizes a harsh chemical, repeated exposure of the nail to a harsh chemical can damage the nail, such as making the nail more brittle.

Techniques for improving nail health are therefore desirable.

### BRIEF SUMMARY

In various aspects, a method for improving nail health of a user may be provided. The method may include providing a composition including an oil phase. The oil phase may include a vasodilator. The vasodilator may be present in a concentration (C_{V}) of, e.g., 0<C_{V}≤5% by weight of the composition. The vasodilator may be a single vasodilator (such as vanillyl butyl ether). The vasodilator may include a plurality of vasodilators. The oil phase may include a non-volatile oil. The non-volatile oil may include one or more vegetable oils, which may be seed oils. The non-volatile oil may be present in a concentration (C_{NVO}) of C_{NVO}≥90% by weight of the composition. The oil phase may include a preservative and an antioxidant. The composition may be in the form of a liquid oil, a thickened oil, or a paste.

The composition may be substantially free of C2-C5 acetates, ketones of less than 6 carbons, and C2-C5 monoalcohols. The composition may be free of film formers. The composition may be substantially free of nitrocellulose. The composition may be free of a colorant. The composition may be free of a fragrance. The composition may be anhydrous.

The method may include applying the composition to a cuticle and/or skin at a base of a nail. The method may include allowing the composition to remain on the cuticle and/or skin at the base of the nail for at least a predetermined period of time. The method may include repeatedly applying the composition to the cuticle and/or skin at the base of the nail at least once a day for a period of at least one week. The composition may be applied to the cuticle and/or skin at the base of the nail using a brush. The composition may be brushlessly applied to the cuticle and/or skin at the base of the nail.

In various aspects, a composition for improving nail health of a user may be provided. The composition may include an oil phase. The oil phase may include a vasodilator. The vasodilator may be present in a concentration (C_{V}) of, e.g., 0<C_{V}≤5% by weight of the composition. The vasodilator may be a single vasodilator (such as vanillyl butyl ether). The vasodilator may include a plurality of vasodilators. The oil phase may include a non-volatile oil. The non-volatile oil may include one or more vegetable oils, which may be seed oils. The non-volatile oil may be present in a concentration (C_{NVO}) of C_{NVO}≥90% by weight of the composition. The oil phase may include a preservative and an antioxidant. The composition may be in the form of a liquid oil, a thickened oil, or a paste.

The composition may be substantially free of C2-C5 acetates, ketones of less than 6 carbons, and C2-C5 monoalcohols. The composition may be free of film formers. The composition may be substantially free of nitrocellulose. The composition may be free of a colorant. The composition may be free of a fragrance. The composition may be anhydrous.

### DETAILED DESCRIPTION

As used herein, the term “about” (a target value) may refer to values that are ±10%, ±8%, ±6%, ±5%, ±4%, ±3%, ±2%, ±1%, or ±0.5% of that target value.

As used herein, the term “substantially free of (a component)” indicates that the systems or compositions contain no appreciable amount of the component, for example, no more than about 1% by weight, or no more than about 0.5% by weight, or no more than about 0.3% by weight, such as no more than about 0.1% by weight, based on the weight of the system or composition comprising the system and/or the oxidizing composition according to embodiments of the disclosure.

As used herein, the term “free” or “completely free of (a component)” indicates that the systems or compositions do not contain the component in any measurable degree by standard means.

The disclosed compositions and techniques may be used to improve nail health. Improving nail health may include, e.g., improving one or more characteristics related to nail health. This may include, e.g., moisturizing skin around the nail, reducing breakage of the nail, reducing brittleness of the nail, conditioning the cuticles, providing barrier protection around the nail plate, and/or providing support for damaged nails.

In various aspects, a method for improving nail health of a user may be provided. The method may include providing a composition for improving nail health of a user.

The composition may include an oil phase. As used herein, the term “include”, “includes”, or “including” (one or more materials) refers to either comprising or consisting of those one or more materials. For example, for a composition “including” an oil phase, the composition may consist of the oil phase, or it may comprise an oil phase.

In a preferred embodiment, the composition may be anhydrous (e.g., free of water). In some embodiments, the composition may be substantially free of water. In some embodiments, the composition may include an aqueous phase. In some embodiments, the aqueous phase may be less than 10% by weight of the composition.

The composition may be in the form of a paste. As used herein, the term “paste” refers to a composition having a consistency that is intermediate between a solid and a liquid, for example, but not limited to, having a viscosity of greater than 5 poises, such as greater than 10 poises at 25° C., and at a shear rate of 1 s⁻¹. This viscosity may be measured using a rheometer or a cone-plate viscometer.

The composition may be in the form of a thickened oil. As used here, the term “oil” refers to a non-aqueous compound, non-miscible in water, liquid, at 25° C. and atmospheric pressure (760 mmHg; 1.013.10⁵ Pa). As used herein, the term “thickened oil” refers to an oil in combination with a thickening or viscosity modifying agent, where the viscosity of the thickened oil is greater than a viscosity of the combination of the base oil(s). For example, the viscosity of a mixture of oil A+oil B+thickening agent C should be greater than the viscosity of a mixture of oil A+oil B. The thickened oil may be unable to flow under its own weight at 20° C. In some embodiments, the thickened oil may have a viscosity of at least 50 Pa-s as measured at a temperature of 25° C. and a shear rate of 1 s⁻¹.

The composition may be in the form of a liquid oil. As used here, the term “liquid oil” refers to an oil that is liquid at room temperature (e.g., 22° C. to 28° C.) preferably also liquid at temperature below room temperature such as below 15° C., 10° C. or 5° C. Preferably, the liquid oil can flow under its own weight at 20° C. In some embodiments, the oil may have a viscosity of less than 50 Pa-s as measured at a temperature of 25° C. and a shear rate of 1 s⁻¹.

### Vasodilator

The oil phase may include a vasodilator. The vasodilator may be any oil soluble or oil miscible cosmetically acceptable vasodilator. Non-limiting examples of such vasodilators includes, e.g., caffeine, cinnamon, menthol, capsaicin, vanillyl butyl ether, ginger oleoresin, L-arginine, niacin, methyl nicotinate, ethyl nicotinate, hexyl nicotinate, etc.

The vasodilator may consist of a single vasodilator. The vasodilator may include a plurality of vasodilators.

The vasodilator may be present in a total concentration (C_{V}) of less than about 5% by weight of the composition. The vasodilator may be present in a total concentration 0<C_{V}≤5% by weight of the composition. In some embodiments, the vasodilator may be present in a total concentration C_{V}≥0.001% by weight of the composition. In some embodiments, the vasodilator may be present in a total concentration C_{V}≥0.01% by weight of the composition. In some embodiments, the vasodilator may be present in a total concentration C_{V}≥0.1% by weight of the composition. In some embodiments, the vasodilator may be present in a total concentration C_{V}≤4.5% by weight of the composition. In some embodiments, the vasodilator may be present in a total concentration C_{V}≤4% by weight of the composition. In some embodiments, the vasodilator may be present in a total concentration C_{V}≤3.5% by weight of the composition. In some embodiments, the vasodilator may be present in a total concentration C_{V}≤3% by weight of the composition. In some embodiments, the vasodilator may be present in a total concentration C_{V}≤2.5% by weight of the composition. In some embodiments, the vasodilator may be present in a total concentration C_{V}≤2.25% by weight of the composition. In some embodiments, the vasodilator may be present in a total concentration C_{V}≤2% by weight of the composition. In some embodiments, the vasodilator may be present in a total concentration C_{V}≤1.75% by weight of the composition. In some embodiments, the vasodilator may be present in a total concentration C_{V}≤1.5% by weight of the composition. In some embodiments, the vasodilator may be present in a total concentration C_{V}≤1.25% by weight of the composition. In some embodiments, the vasodilator may be present in a total concentration C_{V}≤1.00% by weight of the composition. In some embodiments, the vasodilator may be present in a total concentration C_{V}<1.00% by weight of the composition. In some embodiments, the vasodilator may be present in a total concentration C_{V}≤0.75% by weight of the composition. In some embodiments, the vasodilator may be present in a total concentration C_{V}≤0.5% by weight of the composition. In some embodiments, the vasodilator may be present in a total concentration C_{V}≤0.45% by weight of the composition. In some embodiments, the vasodilator may be present in a total concentration C_{V}≤0.4% by weight of the composition. In some embodiments, the vasodilator may be present in a total concentration C_{V}≤0.35% by weight of the composition. In some embodiments, the vasodilator may be present in a total concentration C_{V}≤0.3% by weight of the composition. In one preferred embodiment, the vasodilator may be present in a total concentration 0<C_{V}≤3% by weight of the composition. In one more preferred embodiment, the vasodilator may be present in a total concentration 0<C_{V}≤2.25% by weight of the composition. In one more preferred embodiment, the vasodilator may be present in a total concentration 0<C_{V}≤1.5% by weight of the composition.

In some embodiments with multiple vasodilators, each vasodilator may be present in a total amount of at least about 0.05% by weight of the composition. In some embodiments with multiple vasodilators, each vasodilator may be present in a total amount of at least about 0.1% by weight of the composition. In some embodiments with multiple vasodilators, each vasodilator may be present in a total amount of at least about 0.15% by weight of the composition. In some embodiments with multiple vasodilators, each vasodilator may be present in a total amount of no more than about 1% by weight of the composition. In some embodiments with multiple vasodilators, each vasodilator may be present in a total amount of no more than about 0.75% by weight of the composition. In some embodiments with multiple vasodilators, each vasodilator may be present in a total amount of no more than about 0.5% by weight of the composition. In some embodiments with multiple vasodilators, each vasodilator may be present in a total amount of no more than about 0.4% by weight of the composition. In some embodiments with multiple vasodilators, each vasodilator may be present in a total amount of no more than about 0.3% by weight of the composition. In some embodiments with multiple vasodilators, each vasodilator may be present in a total amount of no more than about 0.2% by weight of the composition.

In some embodiments, when multiple vasodilators are used, each vasodilator may be an agonist against a different receptor. For example, capsaicin and vanillyl butyl ether are agonist for vanilloid receptor (e.g., transient receptor potential vanilloid 1 (TRPV1)), while ethyl nicotinate is used as an agonist for nicotine receptors (e.g., nicotine acetylcholine receptors (nAChRs)).

### Non-Volatile Oil

The oil phase may include a non-volatile oil. As used here, the term “non-volatile” refers to a material (such as an oil) for which the vapor pressure at 25° C. and atmospheric pressure is non-zero and is less than 0.02 mmHg (2.66 Pa) and better still less than 10⁻³ mmHg (0.13 Pa).

The non-volatile oil may include one or more vegetable oils. As used herein, the term “vegetable oil” refers to an oil extracted from a species belonging to the plant kingdom. The vegetable oil may be a seed oil. As used herein, the term “seed oil” refers to oils that are extracted from the seeds of the species from which the vegetable oil is extracted.

Non-limiting examples of seed oils include, e.g., Sunflower oil, cottonseed oil, safflower oil, sesame seed oil, canola oil, grapeseed oil, flaxseed oil, soybean oil, camellia seed oil, meadowfoam seed oil, Buxus chinensis (jojoba) seed oil, and Prunus armeniaca (apricot) kernel oil.

The non-volatile oil may be a single non-volatile oil. The non-volatile oil may be a plurality of non-volatile oils. In one embodiment, the non-volatile oil includes Buxus chinensis (jojoba) seed oil, and Prunus armeniaca (apricot) kernel oil.

The non-volatile oil may be present in a total concentration (C_{NVO}) of at least about 85% by weight of the composition. In some embodiments, the non-volatile oil may be present in a total concentration C_{NVO}≥87.5% by weight of the composition. In some embodiments, the non-volatile oil may be present in a total concentration C_{NVO}≥90% by weight of the composition. In some embodiments, the non-volatile oil may be present in a total concentration C_{NVO}≥92.5% by weight of the composition. In some embodiments, the non-volatile oil may be present in a total concentration C_{NVO}≥95% by weight of the composition. In some embodiments, the non-volatile oil may be present in a total concentration C_{NVO}≥97.5% by weight of the composition.

In some embodiments, when multiple non-volatile oils are present, each non-volatile oil may be present in a concentration of at least about 2.5% by weight of the composition. In some embodiments, when multiple non-volatile oils are present, each non-volatile oil may be present in a concentration of at least about 5% by weight of the composition. In some embodiments, when multiple non-volatile oils are present, one of the non-volatile oils may be present in a concentration of at least about 85% by weight of the composition. In some embodiments, when multiple non-volatile oils are present, one of the non-volatile oils may be present in a concentration of at least about 87.5% by weight of the composition. In some embodiments, when multiple non-volatile oils are present, one of the non-volatile oils may be present in a concentration of at least about 90% by weight of the composition. In some embodiments, when multiple non-volatile oils are present, one of the non-volatile oils may be present in a concentration of at least about 92.5% by weight of the composition.

The non-volatile oil may be substantially free, or free, of a silicone oil. As used herein, the term “silicone oil” refers to an oil containing at least one silicon atom, and in particular containing Si—O groups.

### Preservative

The oil phase may include a preservative. The preservative may be a single preservative. The preservative may be a plurality of preservatives. The preservatives may be oil-soluble or oil-miscible preservatives.

The preservative may include one or more alcohols. The one or more alcohols may include one or more alcohols with a phenoxy terminal group. The alcohol with a phenoxy terminal group may be 2-Phenoxyethan-1-ol (phenoxyethanol). The preservative may include a polyol. The polyol may be, e.g., caprylyl glycol.

The preservative may be present in a total concentration (C_{P}) of less than about 2% by weight of the composition. In some embodiments, the preservative may be present in a total concentration C_{P}≤1.75% by weight of the composition. In some embodiments, the preservative may be present in a total concentration C_{P}≤1.5% by weight of the composition. In some embodiments, the preservative may be present in a total concentration C_{P}≤1.25% by weight of the composition. In some embodiments, the preservative may be present in a total concentration C_{P}≤1% by weight of the composition. In some embodiments, the preservative may be present in a total concentration C_{P}≥0.001% by weight of the composition. In some embodiments, the preservative may be present in a total concentration C_{P}≥0.01% by weight of the composition. In some embodiments, the preservative may be present in a total concentration C_{P}≥0.1% by weight of the composition. In some embodiments, the preservative may be present in a total concentration C_{P}≥0.5% by weight of the composition.

In some embodiments, when more than one preservative is present, each preservative may be present in a concentration of at least about 0.1% by weight of the composition. In some embodiments, when more than one preservative is present, each preservative may be present in a concentration of at least about 0.25% by weight of the composition. In some embodiments, when more than one preservative is present, each preservative may be present in a concentration of at least about 0.5% by weight of the composition.

### Antioxidant

The oil phase may include an antioxidant. The antioxidant may be a single antioxidant. The antioxidant may be a plurality of antioxidants. The antioxidants may be oil-soluble or oil-miscible antioxidants.

Non-limiting examples of antioxidants include ascorbic acid, sodium sulfite, sodium metabisulfite, sodium hydrosulfite, tocopherols, alpha-tocopherol, tocopheryl acetate, panthenol, selenium sulfide, zinc formosulfoxylate, erythorbic acid, isoascorbic acid, thioglycolate salt, magnesium ascorbyl phosphate, ascorbyl glucoside, cysteine, thiourea, thiolactic acid, glyceryl monothioglycolate, thioglycerol, 2,5-dihydroxybenzoic acid, and a mixture thereof. In some cases, the composition maya include at least tocopheryl acetate. composition includes at least ascorbic acid.

The antioxidant may be present in a total concentration (CA) of less than about 2% by weight of the composition. In some embodiments, the antioxidant may be present in a total concentration C_{A}≤1.75% by weight of the composition. In some embodiments, the antioxidant may be present in a total concentration C_{A}≤1.5% by weight of the composition. In some embodiments, the antioxidant may be present in a total concentration C_{A}≤1.25% by weight of the composition. In some embodiments, the antioxidant may be present in a total concentration C_{A}≤1% by weight of the composition. In some embodiments, the antioxidant may be present in a total concentration C_{A}≥0.001% by weight of the composition. In some embodiments, the antioxidant may be present in a total concentration C_{A}≥0.01% by weight of the composition. In some embodiments, the antioxidant may be present in a total concentration C_{A}≥0.1% by weight of the composition. In some embodiments, the antioxidant may be present in a total concentration C_{A}≥0.5% by weight of the composition.

In some embodiments, when more than one antioxidant is present, each antioxidant may be present in a concentration of at least about 0.1% by weight of the composition. In some embodiments, when more than one antioxidant is present, each antioxidant may be present in a concentration of at least about 0.25% by weight of the composition. In some embodiments, when more than one antioxidant is present, each antioxidant may be present in a concentration of at least about 0.5% by weight of the composition.

### Additional Materials

The composition may include one or more additional materials. The one or more additional materials may be present in a total amount less than about 5% by weight of the composition. The one or more additional materials may be present in a total amount less than about 4% by weight of the composition. The one or more additional materials may be present in a total amount less than about 3% by weight of the composition. The one or more additional materials may be present in a total amount less than about 2% by weight of the composition. The composition may be substantially free of additional materials. The composition may be free of additional materials.

### Colorant

The additional materials may include a colorant. In some embodiments, the colorant may be present in a total amount of at least about 0.001% by weight of the composition. In some embodiments, the colorant may be present in a total amount of less than 5% by weight of the composition. In some embodiments, the colorant may be present in a total amount of less than 4% by weight of the composition. In some embodiments, the colorant may be present in a total amount of less than 3% by weight of the composition. In some embodiments, the colorant may be present in a total amount of less than 2% by weight of the composition. In some embodiments, the composition may be substantially free of a colorant. In some embodiments, the composition may be free of a colorant. In some embodiments, the composition may include less than a coloring-effective amount of colorant. For example, if a red colorant is provided, the colorant is not used in a concentration sufficient to cause the product to achieve the desired red color. This amount may vary based on the colorant in question.

The colorant may include one or more dyestuffs chosen from, e.g., liposoluble dyes, pulverulent dyestuffs such as pigments, especially nacres, and glitter flakes, or alternatively coloring polymers.

Among the liposoluble dyes, mention may be made of Sudan Red III (CTFA: D&C Red 17), lutein, quinizarine green (CTFA: D&C Green 6), alizurol purple SS (CTFA: D&C Violet 2), Sudan Brown, D&C Yellow 11, D&C Orange 5, quinoline yellow, curcumin, and carotenoid derivatives such as lycopene, beta-carotene, bixin or capsanthin, and mixtures thereof. The coloring polymers are generally copolymers based on at least two different monomers, at least one of which is a monomeric organic dye. Such polymeric dyes are known to those skilled in the art.

The term “pigments” should be understood as meaning white or colored, mineral or organic particles of any form, which are insoluble in the cosmetic medium, and which are intended to color the cosmetic ink. The term “nacres” should be understood as meaning iridescent particles of any shape, in particular produced by certain mollusks in their shell, or else synthesized.

The pigments may be white, black or colored, and mineral and/or organic. Among the mineral pigments that may be mentioned are titanium dioxide, optionally surface-treated, zirconium oxide or cerium oxide, and also zinc oxide, iron (black, yellow or red) oxide or chromium oxide, manganese violet, ultramarine blue, chromium hydrate and ferric blue, and metal powders, for instance aluminum powder and copper powder. Among the organic pigments that may be mentioned are carbon black, pigments of D&C type and lakes based on cochineal carmine or on barium, strontium, calcium or aluminum. The nacreous pigments may be chosen from white nacreous pigments such as mica coated with titanium or with bismuth oxychloride, colored nacreous pigments such as titanium mica coated with iron oxides, titanium mica coated especially with ferric blue or with chromium oxide, titanium mica coated with an organic pigment and also nacreous pigments based on bismuth oxychloride.

The colorant may include one or more photochromic dyestuffs, especially pigments, i.e. dyestuffs which have the property of changing color when they are irradiated with a light source of a certain frequency, and then of regaining their initial color, or a similar color, when the irradiation is stopped. Among the photochromic dyestuffs, mention may be made especially of: complex mineral photochromic compounds and more particularly doped aluminosilicates, and metal oxides and metal oxide hydrates; and/or photochromic naphthopyran compounds, especially 3H-naphtho[2,1-b]pyrans or 2H-naphtho[1,2-b]pyrans, for instance 3,3-bis(4-methoxyphenyl)-6-morpholino-3H-naphtho[2,1-b]pyran, 3-phenyl-3-(4-morpholinophenyl)-6-morpholino-3H-naphtho[2,1-b]pyran, 3-phenyl-3-(4-piperidinophenyl)-6-morpholino-3H-naphtho[2,1-b]pyran, 3-phenyl-3-(4-piperidinophenyl)-6-carboxymethyl-9-N-dimethyl-3H-naphtho[2,1-b]pyran or 2-phenyl-2-(4-piperidinophenyl)-5-carboxymethyl-9-N-dimethyl-2H-naphtho[1,2-b]pyran.

### Fragrance

The additional materials may include a fragrance. In some embodiments, the fragrance may be present in a total amount of at least about 0.001% by weight of the composition. In some embodiments, the fragrance may be present in a total amount of less than 2% by weight of the composition. In some embodiments, the fragrance may be present in a total amount of less than 1.5% by weight of the composition. In some embodiments, the composition may be substantially free of a fragrance. In some embodiments, the composition may be free of a fragrance.

### Filler

The additional materials may include a filler. The filler may be of organic or inorganic nature. As used herein, the term “filler” generally refers to colorless or white solid particles of any shape, which are in a form that is insoluble or dispersed in the medium of the composition. Inorganic or organic in nature, they make it possible to confer body or rigidity on the composition, and/or softness, and uniformity on the composition.

The filler may be a single filler. The filler may include a plurality of fillers. In some embodiments, the filler may be present in a total amount of at least about 0.001% by weight of the composition. In some embodiments, the filler may be present in a total amount of less than 5% by weight of the composition. In some embodiments, the filler may be present in a total amount of less than 4% by weight of the composition. In some embodiments, the filler may be present in a total amount of less than 3% by weight of the composition. In some embodiments, the filler may be present in a total amount of less than 2% by weight of the composition. In some embodiments, the composition may be substantially free of a filler. In some embodiments, the composition may be free of a filler.

The fillers may be of lamellar, globular or spherical form, or in the form of fibers or in any other form intermediate between these defined forms.

The fillers may or may not be surface-coated, and in particular they may be surface-treated with silicones, amino acids, fluoro derivatives or any other substance that promotes the dispersion and compatibility of the filler in the composition.

Among the fillers that can be used in the compositions according to the present disclosure, mention may be made of talc, mica, kaolin, bentone, precipitated calcium carbonate, magnesium carbonate, magnesium hydrogen carbonate, hydroxyapatite, boron nitride, glass or ceramic microcapsules, composites of silica and of titanium dioxide, such as the TSG series sold by Nippon Sheet Glass, polyamide powders (NYLON® Orgasol from Atochem), poly-β-alanine powders and polyethylene powders, polytetrafluoroethylene powders (TEFLON®), lauroyllysine, starch, hollow polymer microspheres such as Expancel (Nobel Industrie), metal soaps derived from organic carboxylic acids having from 8 to 22 carbon atoms, such as from 12 to 18 carbon atoms, for example zinc stearate, magnesium stearate or lithium stearate, zinc laurate, magnesium myristate, POLYPORE® L 200 (Chemdal Corporation), silicone resin microbeads (for example, Tospearl® from Toshiba), polyurethane powders, in particular powders of crosslinked polyurethane comprising a copolymer, said copolymer comprising trimethylol hexyllactone. In embodiments, it may be a polymer of hexamethylene diisocyanate/trimethylol hexyllactone. Such particles are in particular commercially available, for example under the name PLASTIC POWDER D-400® or PLASTIC POWDER D-800® from the company Toshiki, and mixtures thereof.

### Wax

The additional material may include a wax. The wax may be a single wax. The wax may be a plurality of waxes. In some embodiments, the composition may include one or more soft waxes. In some embodiments, the composition may include one or more hard waxes. In some embodiments, the composition may include a soft wax and a hard wax. As used herein, the term “wax” means a lipophilic compound that is solid at room temperature (25° C.), with a solid/liquid reversible change of state, having a melting point of greater than or equal to 30° C., which may be up to 200° C. and in particular up to 120° C. As used herein, the term “soft wax” refers to waxes which have a melting point of below about 70° C., and preferably, a melting point of below about 60° C. As used herein, the term “hard wax” refers to waxes other than soft waxes—that is, waxes which have a melting point of equal to or greater than about 70° C., and preferably, a melting point of equal to or greater than about 60° C.

In some embodiments, the wax may be present in a total amount of at least about 0.001% by weight of the composition. In some embodiments, the wax may be present in a total amount of less than 5% by weight of the composition. In some embodiments, the wax may be present in a total amount of less than 4% by weight of the composition. In some embodiments, the wax may be present in a total amount of less than 3% by weight of the composition. In some embodiments, the wax may be present in a total amount of less than 2% by weight of the composition. In some embodiments, the composition may be substantially free of a wax. In some embodiments, the composition may be free of a wax.

Non-limiting examples of soft waxes include paraffin wax, ozokerite, synthetic beeswax, beeswax, candelilla wax, hydrogenated jojoba wax, palm butter, sumac wax, polyglyceryl beeswax, siliconyl beeswax, ceresin wax, orange peel wax, silicone resin wax, alkyl dimethicone wax, Berry wax, koster wax, siliconyl candelilla wax, and montan wax.

Non-limiting examples of hard waxes include carnauba wax, microcrystalline wax, polyethylene wax, hydrogenated castor oil, wax AC 540, hydrogenated castor wax, wax AC 400, rice bran wax, Alcohol polyethylene wax, sunflower seed wax, fischer-tropsch wax, Chinese insect wax, and shellac wax.

### Oil Gelling Agent

The additional material may include an oil gelling (or thickening) agent. The oil gelling agent (e.g., a lipophilic thickener) can enhance the stability of the composition even if the oil in the composition is less polar or non-polar. The oil gelling agent may be a single oil gelling agent. The oil gelling agent may be a plurality of an oil gelling agents.

In some embodiments, the wax an oil gelling agent be present in a total amount of at least about 0.001% by weight of the composition. In some embodiments, the oil gelling agent may be present in a total amount of less than 5% by weight of the composition. In some embodiments, the oil gelling agent may be present in a total amount of less than 4% by weight of the composition. In some embodiments, the oil gelling agent may be present in a total amount of less than 3% by weight of the composition. In some embodiments, the oil gelling agent may be present in a total amount of less than 2% by weight of the composition. In some embodiments, the composition may be substantially free of an oil gelling agent. In some embodiments, the composition may be free of an oil gelling agent.

The oil gelling agent (e.g., a lipophilic thickener) can enhance the stability of the composition even if the oil in the composition is less polar or non-polar.

The oil gelling agent may be chosen from gelling agents derived from glutamic acid, gelling agents in polymeric form, and gelling agents in mineral form. The gelling agent includes agents that gel via chemical reticulation and agents that gel via physical reticulation.

N-acyl glutamic acid derivatives may be used as gelling agents derived from glutamic acid. N-acyl glutamic acid derivatives include N-acyl glutamic acid amides and N-acyl glutamic acid esters. In one embodiment, N-acyl glutamic acid amides in which the acyl group represents a C8 to C22 alkyl chain are preferred.

Examples of N-acyl glutamic acid derivatives that may be mentioned include N-lauroyl-glutamic acid diethyl amide, N-lauroyl-glutamic acid dibutyl amide, N-lauroyl-glutamic acid dihexyl amide, N-lauroyl-glutamic acid dioctyl amide, N-lauroyl-glutamic acid didecyl amide, N-lauroyl-glutamic acid didodecyl amide, N-lauroyl-glutamic acid ditetradecyl amide, N-lauroyl-glutamic acid dihexadecyl amide, N-lauroyl-glutamic acid distearyl amide, N-ethylhexanoyl-L-glutamic acid dibutyl amide, N-stearoyl-glutamic acid dibutyl amide, N-stearoyl-glutamic acid dihexyl amide, N-stearoyl-glutamic acid diheptyl amide, N-stearoyl-glutamic acid dioctyl amide, N-stearoyl-glutamic acid didecyl amide, N-stearoyl-glutamic acid didodecyl amide, N-stearoyl-glutamic acid ditetradecyl amide, N-stearoyl-glutamic acid dihexadecyl amide, N-stearoyl-glutamic acid distearyl amide and mixtures thereof, and more preferred is N-lauroyl-glutamic acid dibutyl amide, N-stearyl-glutamic acid dihexyl amide, and mixtures thereof.

The gelling agent may be N-acyl glutamic acid dialkylamide, or N-lauroyl-L-glutamic acid dibutylamide (INCI: dibutyl lauroyl glutamide), manufactured or sold by Ajinomoto under the name GP-1 and N-ethylhexanoyl-L-glutamic acid dibutylamide (INCI: dibutyl ethylhexanoyl glutamide), manufactured or sold by Ajinomoto under the name EB-21.

Lipophilic polyamide polymers may be used as gelling agents in polymeric form. As lipophilic polyamide polymers, mention may be made of polyamides branched with pendent fatty chains and/or terminal fatty chains containing from 12 to 120 carbon atoms and in particular from 12 to 68 carbon atoms, the terminal fatty chains being bonded to the polyamide backbone via ester groups. In particular, mention may be made of the polymers of which the INCI name is “ethylenediamine/stearyl dimer dilinoleate copolymer” and “ethylenediamine/stearyl dimer tallate copolymer”.

Modified clays may be used as gelling agents, examples of which include hectorites modified with an ammonium chloride of a C10 to C22 fatty acid, such as bentonite modified with distearyldimethylammonium chloride, also known as quaternium-18 bentonite, such as the products sold or made under the names BENTONE® 34 organoclay and BENTONE® 38 VCG organoclay by the company Elementis, CLAYTONE® XL rheology additive, CLAYTONE® 34 rheology additive and CLAYTONE® 40 rheology additive sold or made by the company BYK, the modified clays known under the name quaternium-18 benzalkonium bentonites and sold or made under the names CLAYTONE® HT rheology additive, CLAYTONE® GR rheology additive and CLAYTONE® PS rheology additive by the company BYK, the clays modified with stearyldimethylbenzoylammonium chloride, known as stearalkonium bentonites, such as the products sold or made under the names CLAYTONE® APA rheology additive and CLAYTONE® AF rheology additive by the company BYK, and BARAGEL® 24 rheology additive sold or made by the company Elementis. In some embodiments, the composition may be substantially free of modified clays. In some embodiments, the composition may be free of modified clays.

The gelling agent may include, e.g., triesters of a C8-C30 fatty acid. The triesters may be triester(s) of a fatty acid and of mono-or polyglycerol. In some embodiments, the triesters are triesters of fatty acid and of monoglycerol. As used herein, the term “fatty acid” refers to a linear or branched, saturated or unsaturated acid, comprising from 8 to 30 carbon atoms, preferably from 10 to 24 carbon atoms, even more preferentially from 12 to 22 and better still from 16 to 20 carbon atoms, optionally substituted with one or more hydroxyl groups. According to a particular embodiment of the invention, the fatty acid(s) are linear and saturated, substituted with at least one hydroxyl group. The fatty acids may be chosen from oleic acid, lauric acid, palmitic acid, myristic acid, stearic acid, linoleic acid, capric acid and behenic acid, optionally substituted with at least one hydroxyl group, or mixtures thereof. Preferably, the fatty acid(s) are chosen from stearic acid, stearic acids substituted with at least one hydroxyl group, and mixtures thereof, and are more preferentially chosen from stearic acid, 12-hydroxystearic acid, and mixtures thereof, and even better still it is 12-hydroxystearic acid. According to a particular embodiment of the invention, the triester of a C8-C30 fatty acid and of mono-or polyglyceryl is glyceryl tris(12-hydroxystearate). As examples of triester of a C8-C30 fatty acid and of mono-or polyglycerol, mention may be made of glyceryl tri (hydroxystearate) (INCI name: Trihydroxystearin), for instance the product sold as THIXCEN® R non-animal origin rheological additives by the company Elementis.

In some embodiments, the composition may include the vasodilator, the non-volatile oil, the preservative, and the antioxidant. In some embodiments, the composition may include the vasodilator, the non-volatile oil, the preservative, the antioxidant, optionally a colorant, optionally a fragrance, optionally a filler, optionally a wax, optionally an oil gelling agent, and may be substantially free of any other material. In some embodiments, the composition may include the vasodilator, the non-volatile oil, the preservative, the antioxidant, optionally a colorant, optionally a fragrance, optionally a filler, optionally a wax, optionally an oil gelling agent, and may be free of any other material.

In preferred embodiments, the composition may not be configured as a nail polish or nail polish remover composition. Thus, the composition may be substantially free, or free, of one or more materials found in conventional nail polish or nail polish remover compositions.

The composition may be substantially free of acetates. The composition may be free of acetates. In particular, the composition may be substantially free of C2-C5 acetates. The composition may be free of C2-C5 acetates. Such acetates include, e.g., ethyl acetate, butyl acetate, etc.

The composition may be substantially free of ketones. The composition may be free of ketones. The composition may be substantially free of ketones that are liquid at room temperature, such as methyl ethyl ketone, methyl isobutyl ketone, diisobutyl ketone, isophorone, cyclohexanone or acetone. The composition may be free of ketones that are liquid at room temperature. In particular, the composition may be substantially free of ketones of fewer than 6 carbons. The composition may be free of fewer than 6 carbons. Such ketones include, e.g., acetone.

The composition may be substantially free of volatile alcohols. The composition may be free of volatile alcohols. In particular, the composition may be substantially free of C2-C5 monoalcohols. The composition may be free of C2-C5 monoalcohols. Such monoalcohols may include, e.g., ethanol, isopropyl alcohol, isobutyl alcohol, etc.

The composition may be substantially free of a film former. As used herein, the term “film former” generally refers to a polymer or resin that leaves a film on the substrate to which it is applied, for example, after a solvent accompanying the film former has evaporated, absorbed into and/or dissipated on the substrate. The composition may be free of a film former.

The composition may be substantially free of a latex film former. The composition may be free of a latex film former.

The composition may be substantially free of a cellulose derivative film former. The composition may be free of a cellulose derivative film former. Such cellulose derivative film formers may include, e.g., cellulose or chemically modified cellulose such as those obtained by substituting a part or all of the hydroxyl groups in the cellulose with other groups, such as cellulose, nitrocellulose, cellulose acetate, cellulose acetate phthalate, hydroxypropylmethylcellulose phthalate, etc. In one embodiment, the composition may be substantially free of nitrocellulose. In one embodiment, the composition may be free of nitrocellulose.

The method may include applying the composition to a cuticle and/or skin at a base of a nail. The composition may be applied to the cuticle and/or skin at the base of the nail in any appropriate manner. For example, in some embodiments, the composition may be applied to the cuticle and/or skin at the base of the nail using a brush. In some embodiments, the composition may be brushlessly applied to the cuticle and/or skin at the base of the nail.

The method may include allowing the composition to remain on the cuticle and/or skin at the base of the nail for at least a predetermined period of time. The predetermined time may be at least 1 minute. The predetermined time may be at least 5 minutes. The predetermined time may be at least 10 minutes. The predetermined time may be less than 8 hours. The predetermined time may be less than 4 hours. The predetermined time may be less than 2 hours. The predetermined time may be less than 1 hour.

The method may include washing the composition off of the cuticle and/or skin at the base of the nail (e.g., gently using soap and water, or just rinsing with water).

The method may include repeatedly applying the composition to the cuticle and/or skin at the base of the nail at least once a day. The composition may be repeatedly applied for a period of two or more days. The composition may be repeatedly applied for a period of at least one week. The composition may be repeatedly applied for a period of at least two weeks. The composition may be repeatedly applied for a period of at least one month.

In some embodiments, the disclosed compositions and techniques may moisturize skin around the nail. For example, the skin around the nail, prior to use of the disclosed technique, may exhibit lower moisturization characteristics as compared to post-use characteristics. As one specific example, the skin around the nail, prior to use of the disclosed technique, may have a higher transepidermal water loss (TEWL) as compared to the skin after use of the disclosed technique.

In some embodiments, the disclosed compositions and techniques may cause the nails to visually appear longer. In some embodiments, the disclosed compositions and techniques may reduce brittleness of the nails.

### Example 1

The following compositions were formed by combining and mixing the materials at room temperature until homogenous.

| | Ex. 1 | Ex. 2 | Ex. 3 | Control 1 |
|---|---|---|---|---|
| Material | (w/w %) | (w/w %) | (w/w %) | (w/w %) |
| Vasodilators | 0.30 | 0.30 | 0.30 | — |
| Vanillyl Butyl Ether | 0.30 | 0.15 | — | — |
| Ethyl Nicotinate | — | 0.15 | 0.30 | — |
| Non-Volatile Oil | 97.7 | 97.7 | 97.7 | 98.0 |
| Jojoba Seed Oil | 5.0 | 5.0 | 5.0 | 5.0 |
| Apricot Kernel Oil | 92.7 | 92.7 | 92.70 | 93.0 |
| Preservative | 1.0 | 1.0 | 1.0 | 1.0 |
| Antioxidant | 1.0 | 1.0 | 1.0 | 1.0 |

Exemplary formulas (Exs. 1-3) were tested by applying (using a brush) as disclosed herein, and leaving on the skin for 60 minutes, taking readings or measurements at defined points in time (e.g., after 10 minutes, 15 minutes, 20 minutes, 25 minutes, 30 minutes, and 60 minutes). Each of formulas Exs. 1-3 were found to improve at least one characteristic indicative of improved nail health as compared to the control formula (e.g., improved moisturization of skin around the nail, reduced breakage of the nail over time, reduced brittleness of the nail, etc.).

What is claimed is:

1. A method for improving nail health of a user, comprising:
providing a composition including an oil phase, the oil phase including a vasodilator and a non-volatile oil;
applying the composition to a cuticle and/or skin at a base of a nail;
allowing the composition to remain on the cuticle and/or skin at the base of the nail for at least a predetermined period of time.

2. The method of claim 1, further comprising repeatedly applying the composition to the cuticle and/or skin at the base of the nail at least once a day for a period of at least one week.

3. The method of claim 1, wherein the composition is applied to the cuticle and/or skin at the base of the nail using a brush.

4. The method of claim 1, wherein the composition is brushlessly applied to the cuticle and/or skin at the base of the nail.

5. The method of claim 1, wherein the vasodilator is present in a concentration (Cᵥ) of 0<Cᵥ≤5% by weight of the composition.

6. The method of claim 1, wherein the vasodilator is a single vasodilator.

7. The method of claim 1, wherein the vasodilator includes a plurality of vasodilators.

8. The method of claim 1, wherein the non-volatile oil consists of one or more vegetable oils.

9. The method of claim 1, wherein the non-volatile oil is present in a concentration (Cₙᵥₒ) of Cₙᵥₒ≥90% by weight of the composition.

10. The method of claim 1, wherein the composition is substantially free of C2-C5 acetates, ketones of fewer than 6 carbons, and C2-C5 monoalcohols.

11. The method of claim 1, wherein the composition is free of film formers.

12. The method of claim 1, wherein the composition is substantially free of nitrocellulose.

13. The method of claim 1, wherein the composition is free of a colorant.

14. The method of claim 1, wherein the composition is free of a fragrance.

15. The method of claim 1, wherein the composition is anhydrous.

16. The method of claim 1, wherein the oil phase further comprises a preservative and an antioxidant.

17. The method of claim 1, wherein the composition is in the form of a liquid oil, a thickened oil, or a paste.

18. A composition for improving nail health of a user, comprising:
an oil phase, the oil phase including:
a vasodilator;
a preservative;
an antioxidant; and
a non-volatile oil.

19. The composition of claim 18, wherein the vasodilator is a single vasodilator, and the single vasodilator is a vanillyl butyl ether.

20. The composition of claim 18, wherein the composition is substantially free of C2-C5 acetates, ketones of fewer than 6 carbons, and C2-C5 monoalcohols, free of film formers, substantially free of nitrocellulose, and/or free of a colorant.
